(19) United States
(12) Patent Application Publication
(10) Pub. No.: US 2006/0005304 A1
(43) Pub. Date: Jan. 12, 2006

(54) Goggle frame with bendable bridge

(76) Inventor: Terry Chou, Tainan City (TW)

Correspondence Address: CHARLES E. BAXLEY, ESQ., 90 JOHN STREET, THIRD FLOOR, NEW YORK, NY, 10038, US

(21) Appl. No.: 10/888,684

(22) Filed: Jul. 8, 2004

(51) Int. Cl. A61F 9/02

(52) U.S. Cl. 2/426

(57) ABSTRACT

A goggle frame includes two ring portions made of a rigid material with stretching resistance and a bridge integrally formed between the ring portions as a one-piece member. The ring portions hold two lenses, respectively. The bridge is stretchable and bendable, allowing adjustment of a length of the bridge to meet the distance between the eye sockets of the user. The bridge has a color the same as or different from that of the ring portions.

### BACKGROUND OF THE INVENTION

1. Field of the Invention

The present invention relates to a goggle frame. In particular, the present invention relates to a goggle frame with a bendable bridge.

2. Description of the Related Art

FIGS. 12 and 13 of the drawings illustrate a pair of conventional swimming goggles comprising two rigid lenses 10′, a slightly flexible bridge 11′, two soft padding members 12′, and a head strap 13′. Each rigid lens 10′ includes an annular portion 101′ and a perimeter portion 104′. Each rigid lens 10′ further includes an engaging hole 102′ in an inner end thereof and an engaging hole 103′ in an outer end thereof for respectively engaging with the bridge 11′ and an associated end of the head strap 13′. The bridge 11′ is substantially U-shaped and includes a plurality of teeth 111′ in an inner surface thereof, with two limbs (not labeled) of the bridge 11′ being engaged with the engaging holes 102′ of the lenses 10′. Each soft padding member 12′ is made of soft material and engaged with the annular portion 101′ and the perimeter portion 104′ of an associated lens 10′, with the soft padding member 12′ being in contact with an associated eye socket of a user, providing wearing comfort.

The bridge 11′ may have a color different from that of the lenses 10′ to provide various color combination. However, since the bridge 11′ is separate from the lenses 10′, the lenses 10′ could not be reliably fixed in a horizontal position, failing to provide an aesthetically pleasing appearance, as shown in FIG. 13. Further, the bridge 11′ may fall when the head strap 13′ exerts a relatively large rearward pulling force to the lenses 10′.

FIGS. 14 and 15 illustrate another pair of conventional swimming goggles comprising two rigid lenses 20′, a frame with two ring portions 21′, a bridge 22′, two padding members 23′, and a head strap 24′. Each lens 20′ is mounted in a front portion of an associated ring portion 21′. The frame, the bridge 22′, and the padding members 23′ are formed with the same material, with each ring portion 21′ having an annular portion 211′ for contacting with an associated eye socket of a user and with each ring portion 21′ having an annular groove 212′ for receiving a perimeter edge of an associated lens 20′. A flexible plate 221′ is embedded into the bridge 22′ to provide an aesthetically pleasing appearance.

However, since each padding member 23′ must be made of a soft material so as to be in intimate contact with the associated eye socket of the user, the frame has to be manufactured with the same soft material and is thus apt to deform when the head strap 24′ is pulled, as shown in FIG. 15. The annular portion 211′ deforms largely, twisting the annular groove 212′. As a result, water leakage may occur in the area between each lens 20′ and the associated ring portion 21′. Further, the bridge 22′ and the frame have the same color and thus fail to provide various colorful combinations. Further, a flexible plate 221′ is required in the bridge 22′ to avoid exerting a pressure to the nose of the user due to over deformation of the bridge 22′. Additional cost for the flexible plate 221′ is required.

FIGS. 16 and 17 illustrate a further pair of conventional swimming goggles comprising two lenses 30′, a frame with two ring portions 31′, a bridge 32′, two padding members 33′, and a head strap 34′. Each lens 30′ is made of a rigid material and includes an annular portion 301′ having two engaging holes 302′ (only one is shown). The ring portions 31′ and the bridge 32′ are integrally formed with a rigid material, with each ring portion 31′ including two engaging members 311′. Each rigid lens 30′ is forcibly inserted into an associated ring portion 31′, with the engaging members 311′ being engaged with the engaging holes 302′. Each padding member 33′ is made of a soft material and mounted to a rear side of an associated lens 30′ for intimate contact with an associated eye socket of a user. Two ends of the head strap 34′ are respectively engaged with two sides of the frame.

Since each ring portion 31′ is rigid and thus has excellent stretching resistance, undesired disengagement of the associated lens 30′ resulting from deformation of the ring portion 31′ can be avoided. However, since the curvature of the rigid bridge 32′ is fixed, the distance between the lenses 30′ could not be adjusted for different users. Further, the bridge 32′ and the frame have the same color and thus fail to provide various colorful combinations. Further, it is difficult to stretch the rigid ring portions 31′ with a low stretching flexibility when mounting the lenses 30′. The assembling is not convenient and the engaging members 311′ break easily.

### SUMMARY OF THE INVENTION

In accordance with a first aspect of the present invention, a goggle frame comprises two ring portions made of a rigid material with stretching resistance and a bridge integrally formed between the ring portions as a one-piece member. The ring portions hold two lenses, respectively. The bridge is stretchable and bendable, allowing adjustment of a length of the bridge to meet the distance between the eye sockets of the user.

The bridge has a color the same as or different from that of the ring portions.

Each ring portion may include at least one engaging member, and each lens may include at least one engaging groove for engaging with the engaging member.

In an embodiment of the invention, each ring portion includes an inner side having a notch to allow easy insertion of the associated lenses.

In another embodiment of the invention, each ring portion includes an inner side enveloped by the bridge.

In a further embodiment of the invention, each ring portion includes an inner side having a through-hole through which the bridge extends.

In still another embodiment of the invention, each ring portion includes an inner side having a protrusion enveloped by the bridge.

In yet another embodiment of the invention, each ring portion includes a head strap engaging portion.

Other objectives, advantages, and novel features of the invention will become more apparent from the following detailed description when taken in conjunction with the accompanying drawings.

### BRIEF DESCRIPTION OF THE DRAWINGS

FIG. 1 is a perspective view of a goggle frame in accordance with the present invention.

FIG. 2 is a top sectional view of the goggle frame in accordance with the present invention.

FIG. 3 is an exploded perspective view of a pair of swimming goggles with the goggle frame in accordance with the present invention.

FIG. 4 is a sectional view of the pair of swimming goggles in FIG. 3.

FIG. 5 is a top view of the pair of swimming goggles in FIG. 3.

FIG. 6 is a view similar to FIG. 2, illustrating a modified embodiment of the goggle frame in accordance with the present invention.

FIG. 7 is a front view of another modified embodiment of the goggle frame in accordance with the present invention.

FIG. 8 is a view illustrating assembling of the goggle frame in FIG. 7.

FIG. 9 is a top view illustrating a further modified embodiment of the present invention.

FIG. 10 is a top sectional view illustrating still another modified embodiment of the present invention.

FIG. 11 is a top sectional view illustrating yet another modified embodiment of the present invention.

FIG. 12 is an exploded perspective view of a pair of conventional swimming goggles.

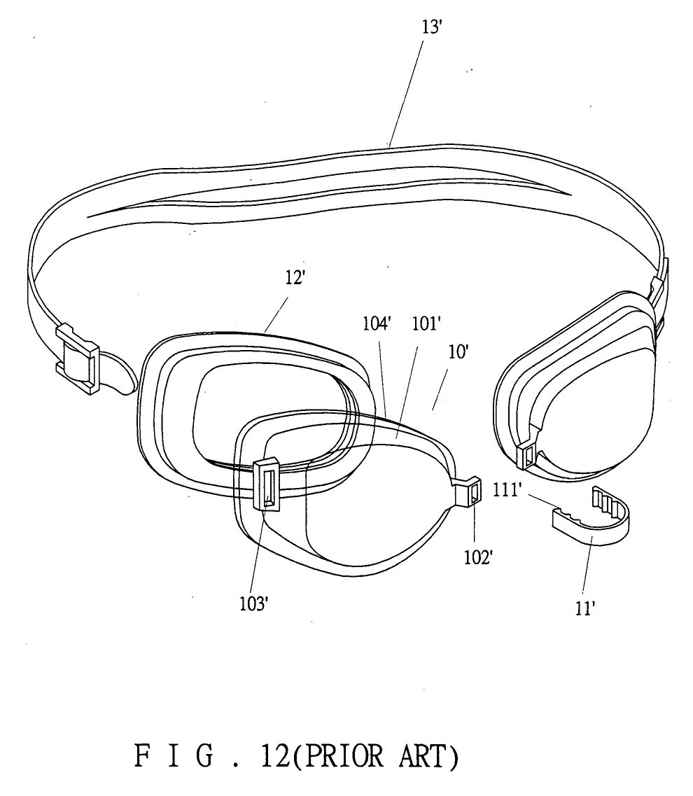

FIG. 13 is a front view of the pair of conventional swimming goggles in FIG. 12.

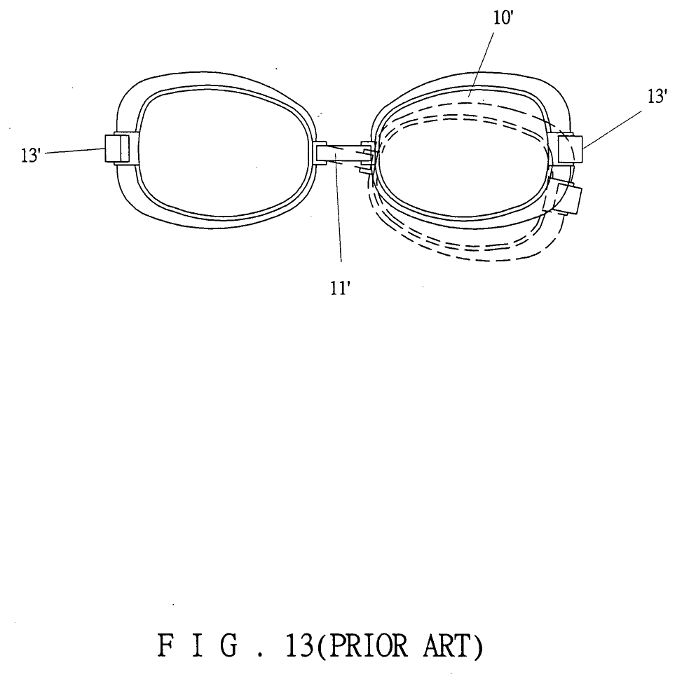

FIG. 14 is a top sectional view of another pair of conventional swimming goggles.

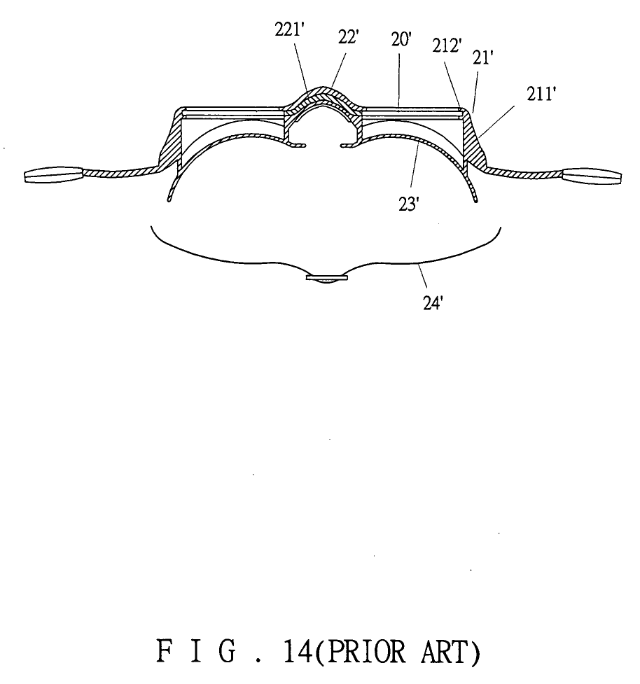

FIG. 15 is an enlarged view of a portion of the pair of conventional swimming goggles in FIG. 14.

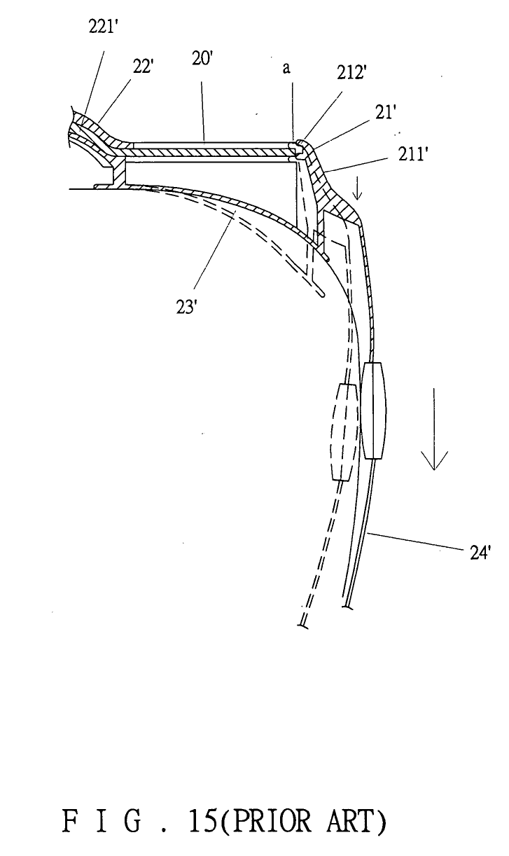

FIG. 16 is an exploded perspective view of a further pair of conventional swimming goggles.

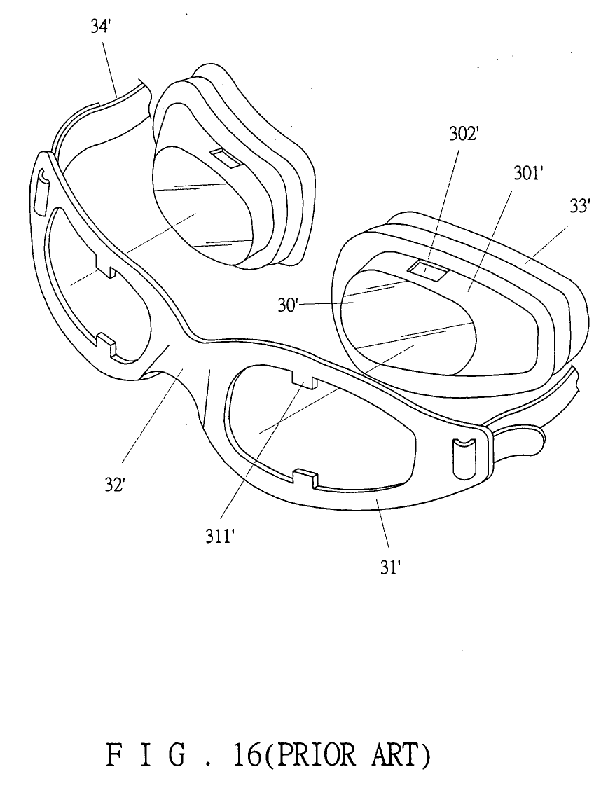

FIG. 17 is a perspective view of the pair of conventional swimming goggles in FIG. 16.

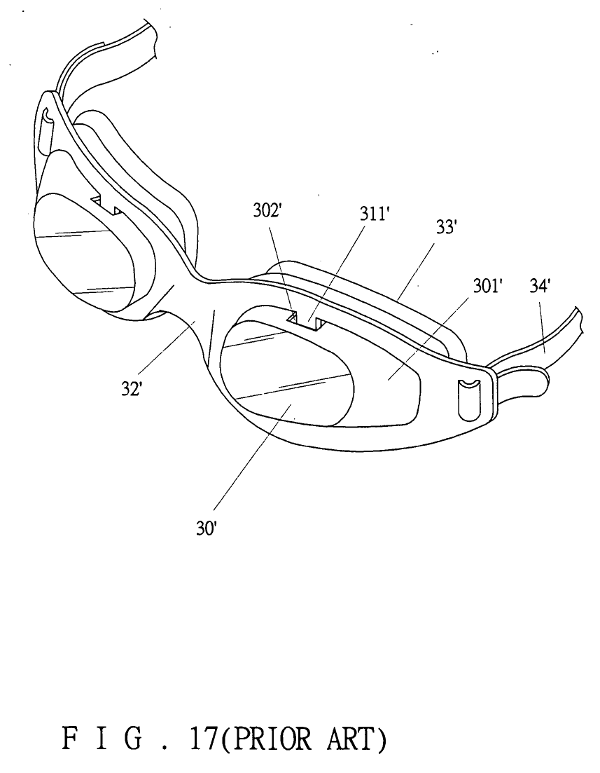

### DETAILED DESCRIPTION OF THE PREFERRED EMBODIMENTS

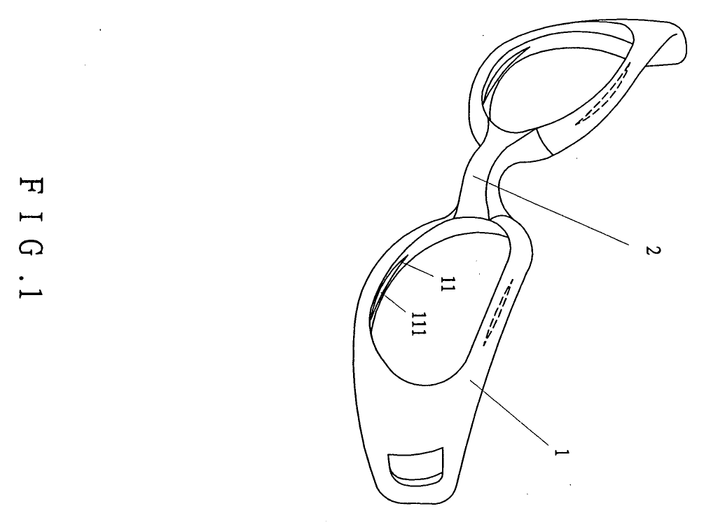

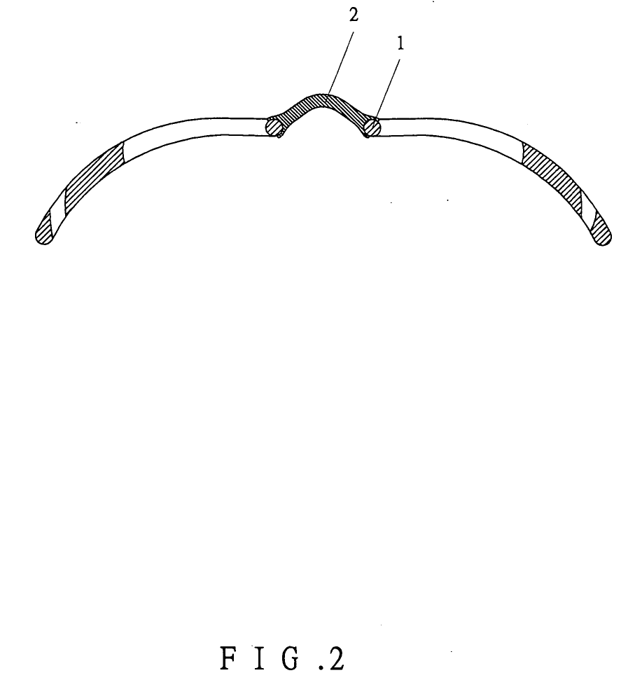

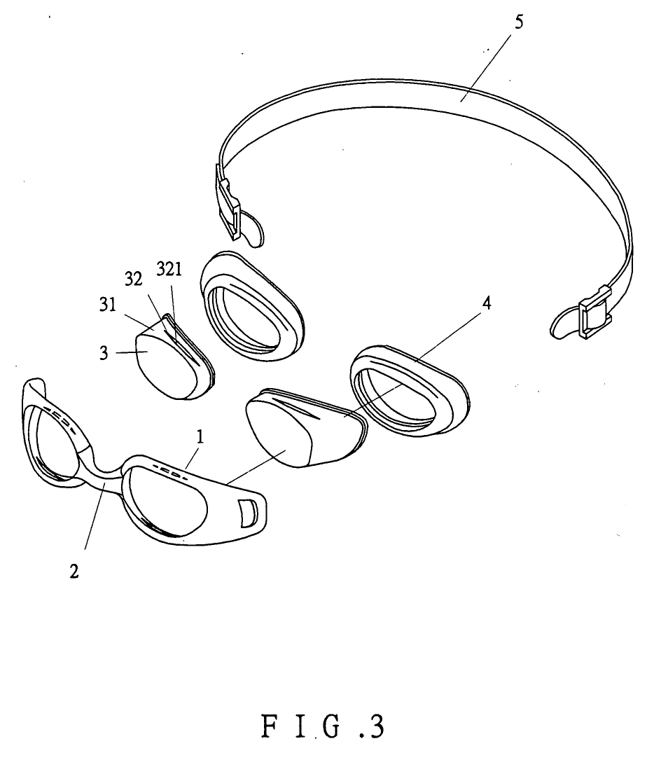

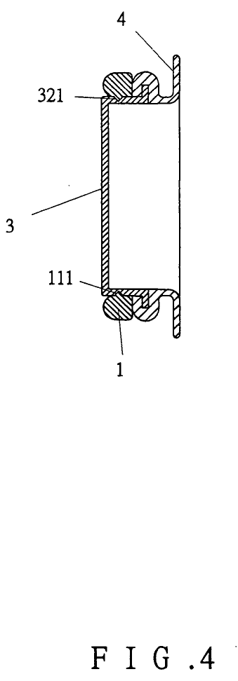

Referring to FIGS. 1 through 4, a pair of swimming goggles with a goggle frame in accordance with the present invention comprises two ring portions 1, a bridge 2, two lenses 3, two padding members 4, and a head strap 5. Each ring portion 1 is made of a rigid material with excellent stretching resistance and annular for receiving an associated lens 3. Further, each ring portion 1 includes at least one engaging portion 11 (two in this embodiment) with an engaging member 111.

The bridge 2 is made of stretchable and bendable material with a desired color. In manufacture, an inner side of each rigid ring portions 1 is placed in a mold (not shown), and molten plastic material for the bridge 2 is poured into the mold forming the bridge 2, with two ends of the bridge 2 being integrally formed with the inner sides of the rigid ring portions 1 to form a goggle frame in accordance with the present invention.

Each lens 3 is made of a rigid material with excellent stretching resistance and includes an annular portion 31. Further, each lens 3 includes at least one engaging portion 32 (two in this embodiment) with an engaging groove 321 for engaging with the engaging member 111 of an associated ring portion 1.

Each padding member 4 is mounted to a rear side of an associated lens 3 and made of a soft material for providing comfortable intimate contact with the associated eye socket of the user. The head strap 5 is stretchable and has two ends respectively engaged with outer sides of the ring portions 1.

Since the ring portions 1 and the bridge 2 are integrally formed as a one-piece member, a goggle frame with a reliable integral structure is provided. Further, the bridge 2 may have a color different from that of the ring portions 1, various color combination can be obtained.

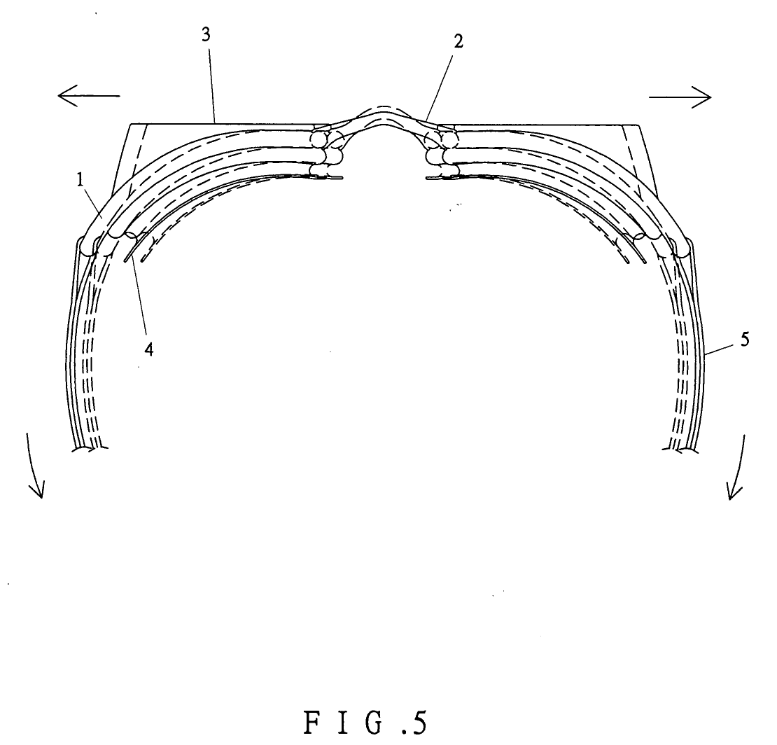

Referring to FIG. 5, when in use, the bendable goggle frame 2 may be bent to adjust its overall length in response to the distance between the eye sockets of the user. Further, the ring portions 1 and lenses 3 with excellent stretching resistance prevent excessive deformation of the goggle frame, allowing reliable intimate contact between the padding members 4 and the eye sockets of the user and thus providing improved waterproof effect.

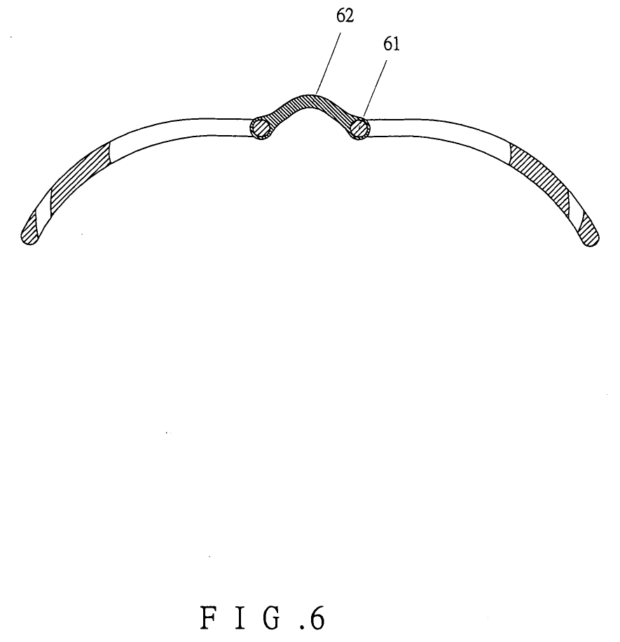

In a modified embodiment of the invention shown in FIG. 6, the bridge (now designated by 62) is annular and envelopes the inner sides of the ring portions 61, providing improved bonding relationship between the ring portions 6 and the bridge 62.

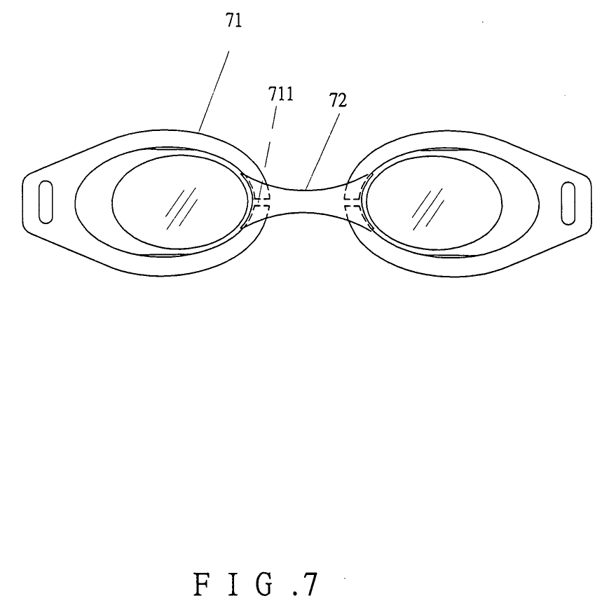

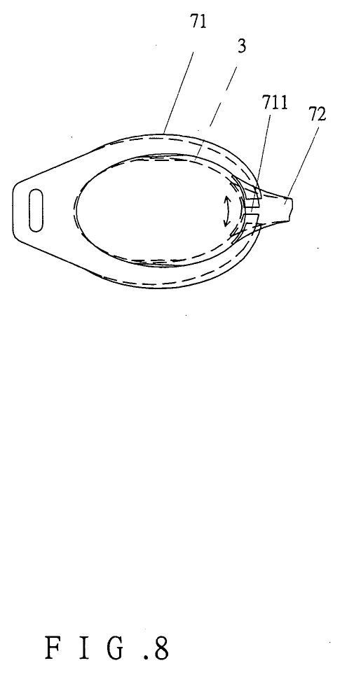

In another modified embodiment of the invention shown in FIGS. 7 and 8, each ring portion (now designated by 71) includes a notch 711 in the inner side thereof. As illustrated in FIG. 8, when mounting the rigid lenses 3 into the rigid ring portions 71, each rigid ring portion 71 may be stretched outward at the notch 711 to allow easy insertion of the associated rigid lens 3 into the rigid ring portion 71.

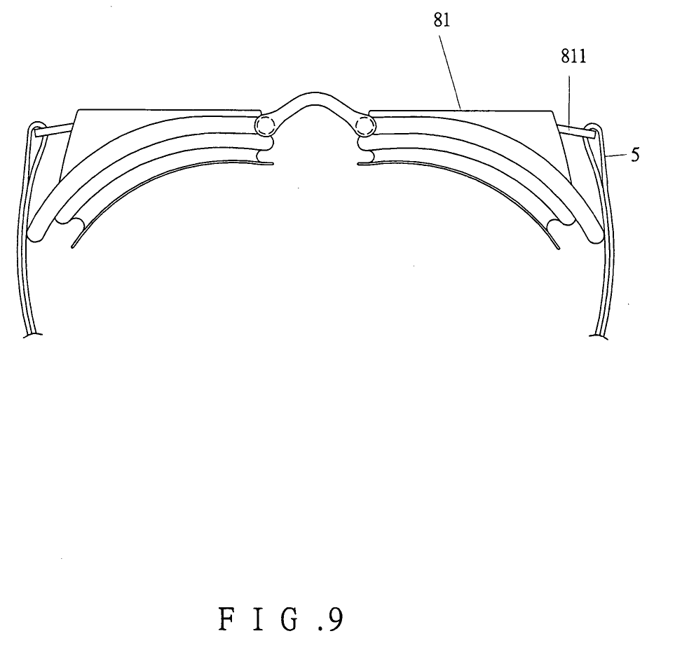

In a further modified embodiment of the invention shown in FIG. 9, each lens (now designated by 81) includes a head strap engaging portion 811 for engaging with an associated end of the head strap 5.

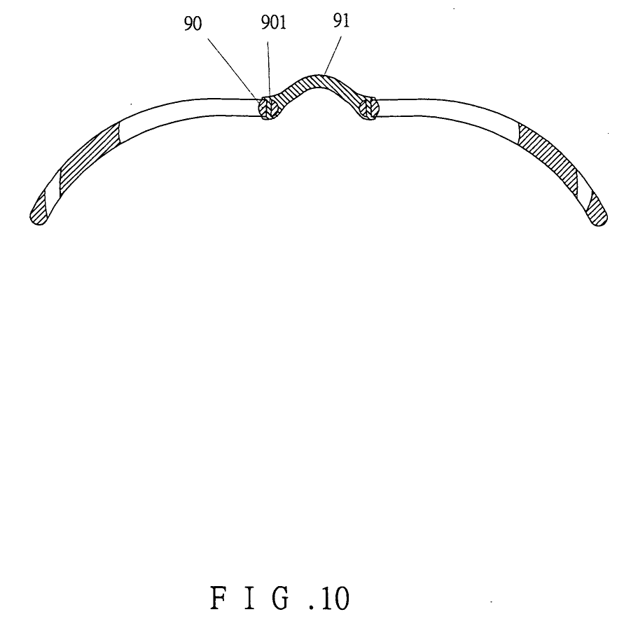

In still another modified embodiment of the invention shown in FIG. 10, each ring portion (now designated by 90) includes a through-hole 901 in the inner side thereof. When placed in the mold (not shown) for forming the goggle frame, the molten plastic material fills the through-hole 901 in each ring portion 90. Thus, the bridge (now designated by 91) extends through the through-holes 901 of the ring portions 90 after formation, providing reliable bonding between the bridge 91 and the ring portions 90.

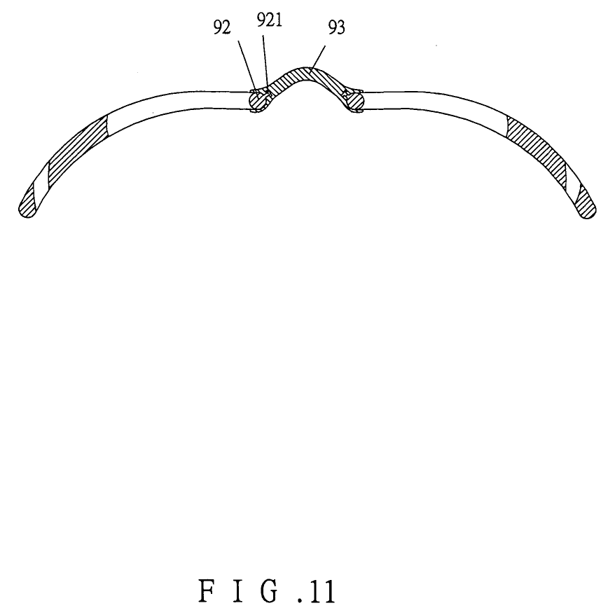

In yet another modified embodiment of the present invention shown in FIG. 11, each ring portion (now designated by 92) includes a protrusion 921 on the inner side thereof. When placed in the mold (not shown) for forming the goggle frame, the molten plastic material envelopes the protrusions 921, providing reliable bonding between the bridge (now designated by 93) and the ring portions 90.

Although specific embodiments have been illustrated and described, numerous modifications and variations are still possible without departing from the essence of the invention. The scope of the invention is limited by the accompanying claims.

What is claimed:

1. A goggle frame comprising:
two ring portions made of a rigid material with stretching resistance, the ring portions being adapted to hold two lenses; and
a bridge integrally formed between the ring portions as a one-piece member, the bridge being stretchable and bendable, allowing adjustment of a length of the bridge.

2. The goggle frame as claimed in claim 1, wherein the bridge has a color the same as that of the ring portions.

3. The goggle frame as claimed in claim 1, wherein the bridge has a color different from that of the ring portions.

4. The goggle frame as claimed in claim 1, wherein each said ring portion includes an inner side having a notch.

5. The goggle frame as claimed in claim 1, wherein each said ring portion includes an inner side enveloped by the bridge.

6. The goggle frame as claimed in claim 1, wherein each said ring portion includes an inner side having a through-hole through which the bridge extends.

7. The goggle frame as claimed in claim 1, wherein each said ring portion includes an inner side having a protrusion enveloped by the bridge.

8. The goggle frame as claimed in claim 1, wherein each said ring portions includes a head strap engaging portion.

9. The goggle frame as claimed in claim 1, further including two lenses respectively mounted in the ring portions, each said ring portion including at least one engaging member, each said lens including at least one engaging groove with said at least one engaging member.

10. The goggle frame as claimed in claim 1, further comprising a slot portion formed in said ring portions thereby impacting a degree of flexibility to said ring portions.

11. A goggle from comprising:
two ring portions made of a rigid material with stretching resistance, the ring portions being adopted to hold two lenses, with the ring portions each having an inner portion, and
a bridge integrally formed between the ring portions as a one piece member, the bridge being stretchable and bendable, allowing adjustment of a length of the bridge and with the bridge disposed abutting said inner portions of said ring portions.
